(19) United States
(12) Patent Application Publication
(10) Pub. No.: US 2012/0244134 A1
(43) Pub. Date: Sep. 27, 2012

(54) PROCESS FOR PREPARING AQUEOUS DISPERSIONS CONTAINING HIGH CONCENTRATION OF NANO/SUBMICRON, HYDROPHOBIC, FUNCTIONAL COMPOUNDS

(76) Inventors: Ru-Yin CHEN, Hsinchu City (TW); Chung-Jen CHEN, Hsinchu City (TW); Yi-Jie TSAI, Hsinchu City (TW); Jia-Jiu WU, Hsinchu City (TW); Chih-Ping HUANG, Hsinchu City (TW); Chung-Liang CHU, Hsinchu City (TW)

(21) Appl. No.: 13/186,176

(22) Filed: Jul. 19, 2011

(30) Foreign Application Priority Data
Mar. 21, 2011 (TW) 100109581

(51) Int. Cl. A61K 38/43; A61K 31/07; A61K 31/59; A61K 31/01; A61K 31/355; A61K 31/015; A61K 31/047; A61K 31/357; A61K 31/7048; A61K 9/14; A23L 3/3553; A61K 47/24; A61K 31/12; B82Y 5/00

(52) U.S. Cl. 424/94.1; 514/785; 514/725; 514/167; 514/762; 514/458; 514/682; 514/763; 514/729; 514/685; 514/452; 514/27; 424/400; 426/654; 426/648; 977/773; 977/915

(57) ABSTRACT

The present invention provides a process for preparing an aqueous dispersion containing a high concentration of nano/submicron, hydrophobic, functional compounds. The process is carried out by using a complex stabilizer having an HLB value of about 10 to about 17, comprising lecithin and at least one non-phospholipid selected from polysorbate, sucrose ester, and polyglycerol fatty acid ester; selecting a specific weight ratio of the hydrophobic functional compounds and the stabilizer; and using homogenization technique, media milling technique, and/or centrifugal technique. The aqueous dispersion containing a high concentration of nano/submicron, hydrophobic, functional compound produced by the process of the invention has stable dispersibility and improved bioavailability, and can be applied to the fields of foods and pharmaceuticals.

### FIELD OF THE INVENTION

The present invention relates to a process for preparing an aqueous dispersion containing a high concentration of nano/submicron, hydrophobic, functional compounds as well as an aqueous dispersion containing a high concentration of nano/submicron, hydrophobic, functional compounds obtained therefrom.

### BACKGROUND OF THE INVENTION

In the field of functional foods and nutraceuticals, a good deal of research and development is directed to the objective of modifying hydrophobic functional components to be hydrophilic so that they can be advantageously added to a solution of foods, thereby increasing their applicability in the field of foods.

Most prior art references show that a stabilizer must be present in an amount of 1.4-50% (w/v), preferably 10-20% (w/v), on the basis of a solution, so that a desired effect can be achieved. Please refer to references 1 to 8. Consequently, the amount of stabilizer required is typically greater than the amount of extracted functional materials dispersed, leading to the commonly encountered problem that a large amount of stabilizers is taken when taking in functional substances.

Techniques for homogenously mixing an oil/water phase and a stabilizer are known in the art. For example, it can be performed by the steps of: dissolving stabilizers and hydrophobic functional compounds with an organic solvent, homogenously mixing these materials by blending, homogenization, ultrasonic processing, etc., and removing the solvent to achieve the objective of micro-emulsification. Please refer to references 4-7, 9-15. Alternatively, apparatuses such as homogenizers, high-speed homogenizers (see references 1, 10-11), high-pressure homogenizers (see reference 1), ultrasonic processors (see references 2, 12), ball grinding millers (see reference 6), and media millers (see references 3, 17) can be used to achieve a homogenously emulsifying efficacy.

Because conventional methods usually prepare nano/submicron particles via micro-emulsification and anti-solvent precipitation, they tend to have disadvantages such as requiring complicated manufacturing procedures, a plurality of solvents or a large quantity of emulsifiers. They also tend to be inapplicable to non-pure substances, and achieve relatively low yield, thus presenting significant challenges to industrial practicability.

Functional food materials include vitamins, carotenenoid terpenoids, polyphenolics, etc. Among these materials, those which are difficult to dissolve in water include lipid-soluble vitamins (for example, vitamins A, D, E, K, and CoQ10), carotenenoid terpenoids (for example, lycopene, carotene, lutene, zeaxanthin, etc.), non-flavonoid polyphenolics which belong to hydrophobic polyphenolics, for example, curcumin, and flavonoides polyphenolics, for example, silymarin and isoflavonoid. Please refer to reference 18. Even if the aforementioned functional food materials which are difficult to dissolve in water are subjected to certain manufacturing procedures, only 0.1-2% (w/v) of the functional components are homogenously and stably dispersed in an aqueous solution. Please refer to references 1, 2, 19. Moreover, functional food materials which are difficult to dissolve in water are also difficult to absorb. Please refer to references 20-21. Despite their potential value in health care, their inability to dissolve in water constrains the applicability of such functional food materials.

Therefore, a technique is sought for preparing an aqueous dispersion of hydrophobic, functional compounds which can preclude the need for organic solvents, reduce the amount of stabilizers required, increase the concentration of functional compounds homogenously dispersed in water, and enhance bioavailability of functional compounds.

### SUMMARY OF THE INVENTION

The present invention achieves an aqueous dispersion containing a high concentration of nano/submicron, hydrophobic, functional compounds by using a complex stabilizer having an HLB value of about 10 to about 17, comprising lecithin and at least one non-phospholipid selected from polysorbate, sucrose ester, and polyglycerol fatty acid ester; selecting a specific weight ratio of the hydrophobic functional compounds and the stabilizer; and using homogenization technique, media milling technique, and/or centrifugal technique. The aqueous dispersion containing a high concentration of nano/submicron, hydrophobic, functional compounds of the present invention has stable dispersibility and improved bioavailability, and can be applied to the fields of foods and pharmaceuticals. The process of the present invention has the advantages that it does not require organic solvents, significantly reduces the required quantity of stabilizers, and increases the concentration of nano/submicron, hydrophobic, functional compounds in an aqueous dispersion. Thus, the present invention addresses the long-standing problem encountered in the art that a large amount of stabilizers is taken when taking in functional substances thereby limiting the potential concentration of functional compounds.

### DETAILED DESCRIPTION OF THE INVENTION

According to an embodiment of the present invention, a process for preparing an aqueous dispersion containing a high concentration of nano/submicron, hydrophobic, functional compounds is provided by the following steps:
formulating a complex stabilizer and water into an aqueous solution containing a complex stabilizer, wherein said complex stabilizer has an HLB value of about 10 to about 17 and comprises lecithin and at least one non-phospholipid selected from polysorbate, sucrose ester, and polyglycerol fatty acid ester;
Incorporating hydrophobic, functional compounds into the aqueous solution containing a complex stabilizer to form a non-homogenously mixed liquid wherein the weight ratio of the hydrophobic, functional compounds to the complex stabilizer is from 2:1 to 10:1;
Subjecting the non-homogenously mixed liquid to a homogenization pretreatment to form a homogenously mixed liquid;
Subjecting the homogenously mixed liquid to nano-grade wet grinding to form an aqueous dispersion; and
Optionally, subjecting the aqueous dispersion from nano-grade wet grinding to a centrifugal step and collecting the supernatant.

The term “mixed liquid” as used herein refers to a liquid in which solutes are precipitated and separated from the solution after the liquid is stored for a period of time. The term “non-homogenously mixed liquid” as used herein refers to a liquid in which solutes are added to the solution only by stirring so that they are present in the form of massed particles which are difficult to homogenously disperse in a solution, which leads to precipitation and separation of the liquid. The term “homogenously mixed liquid” as used herein refers to a liquid in which solutes are added by stirring followed by homogenization so that the solutes are homogenously distributed for a period of time before eventually precipitating and separating over time.

The term “dispersion” as used herein refers to a liquid in which solutes remain steadily and homogenously distributed in the liquid after the liquid is stored for a period of time.

The term “high concentration” as used herein refers to the condition that the concentration (w/v) of nano/submicron, hydrophobic, functional compounds in the aqueous dispersion obtained from nano-grade wet grinding is from about 1 mg/mL (0.1%) to about 200 mg/mL (20%), preferably, from about 10 mg/mL (1%) to about 150 mg/mL (15%). After the aqueous dispersion is further subjected to centrifugal and collecting steps, it contains nanoparticles in a proportion of about 20% to about 85% (w/w), preferably, about 40% to about 85% (w/w), and more preferably, about 60% to about 85% (w/w), on the basis of the total particles.

The term “nano” as used herein refers to particle size less than 300 nm. The phrase “submicron” as used herein refers to particle size less than 2,000 nm. Because most foods are organic materials, for which test data and standards are relatively sparse, the terms “nano” and “submicron” have broader meanings in the field of foods.

The term “HLB value” (hydrophilic-lipidphilic balance value) as used herein refers to the level of balance between the size and strength of hydrophilic groups and lipidphilic groups of surfactants.

The aqueous solution containing a complex stabilizer of the present invention can be prepared by any conventionally known techniques. For example, it can be prepared by a method comprising separately melting lecithin and at least one non-phospholipid selected from polysorbate, sucrose ester, and polyglycerol fatty acid ester in a weight ratio of about 1:99 to about 99:1, preferably, about 15:85 to about 85:15, via heating, homogenously stirring the non-phospholipid and the lecithin to form a complex stabilizer having an HLB value of about 10 to about 17, preferably, about 10 to about 15; incorporating the complex stabilizer into water in an amount of about 0.01% to about 10.0% (w/v), preferably, about 0.1% to about 4.5% (w/v), relative to the volume of water; and homogenously stirring the same so as to form an aqueous solution containing a complex stabilizer. Alternatively, it can be prepared by a method comprising separately incorporating lecithin and at least one non-phospholipid selected from polysorbate, sucrose ester, and polyglycerol fatty acid ester in a weight ratio of about 1:99 to about 99:1, preferably, about 15:85 to about 85:15, into water in a total amount of the lecithin and the non-phospholipid of about 0.01% to about 10.0% (w/v), preferably, about 0.1% to about 4.5% (w/v), relative to the volume of water; and heating and homogeneously stirring the same to form an aqueous solution containing a complex stabilizer. The complex stabilizer of the aqueous solution containing a complex stabilizer has an HLB value of about 10 to about 17, preferably, about 10 to about 15.

The term “lecithin” as used herein refers to a substance extracted from soybeans, which can be further modified. The substance essentially comprises components such as phosphatidylcholine, phosphatidylethanolamine, phosphatidylinositol, and phosphatidylserine. The present invention uses lecithin having an HLB value of about 4 to about 10, preferably, about 8 to about 10.

The present invention uses polysorbates having an HLB value of about 11 to about 17. Specific examples of polysorbates include but are not limited to polysorbate 20 having an HLB value of 16.7, polysorbate 80 having an HLB value of 15, polysorbate 65 having an HLB value of 10.5, and polysorbate 60 having an HLB value of 14.9.

The term “sucrose ester” as used herein refers to a sucrose fatty acid ester formed from the esterification of sucrose and fatty acid. The fatty acid can be, for example, oleic acid, stearic acid, and palmitic acid. The present invention uses sucrose ester having an HLB value of about 11 to about 17.

The term “polyglycerol fatty acid ester” as used herein refers to an ester formed from the esterification of polyglycerol and fatty acid. The fatty acid can be, for example, oleic acid, stearic acid, and palmitic acid. The present invention uses polyglycerol fatty acid ester having an HLB value of about 11 to about 17.

The term “hydrophobic, functional compounds” as used herein refers to functional compounds which almost cannot dissolve in water. The term “almost cannot dissolve in water” as used herein refers to that the compounds have a solubility of less than 10⁻⁴ M in water. Examples of hydrophobic, functional compounds include lipid-soluble vitamins, for example, vitamins A, D, E, K, and CoQ10, carotenenoid terpenoids, for example, lycopene, carotene, lutene, and zeaxanthin, etc., non-flavonoides polyphenolics which belong to hydrophobic polyphenolics, for example, curcumin and sesamin, and flavonoides polyphenolics, for example, silymarin, isoflavonoid, and hesperidin, and mixtures thereof. Any other hydrophobic, functional compounds which are known to be useful in the fields of functional foods and nutraceuticals are applicable to the present invention.

In a preferred embodiment of the present invention, the amount of hydrophobic, functional compounds is about 0.1% to about 20% (w/v), preferably, about 1% to about 15% (w/v); the amount of the complex stabilizer is about 0.01% to about 10% (w/v), preferably, about 0.1% to about 4.5% (w/v). The aforementioned amounts are weighed relative to the volume of water.

In the present invention, the weight ratio of the hydrophobic, functional compounds to the complex stabilizer is about 2:1 to about 10:1, preferably, about 3:1 to about 8:1.

In the process of the present invention, the homogenization pretreatment of the aqueous dispersion can be carried out by any conventional means known in the art. For example, it can be carried out by using a homogenizer or an ultrasonic processor. Homogenizers of any blends known in the art are applicable to the present invention. For example. Pro-400 Pro Scientific Inc. manufactured by Oxford CT. U.S.A. can be used. Ultrasonic processors of any blends known in the art are applicable to the present invention. For example. Sonicator 4000 Ultrasonic Liquid Processors manufactured in the U.S.A. can be used.

Nano-grade wet grinders of any blends known in the art are applicable to the process of the present invention. For example, the nano-grade wet grinding step can be carried by using a nano-grade wet grinder commercially available under the trade name “MiniCer” (manufactured and sold by Netzsch-Feinmahltechnik GmbH, Selb, Germany) and a nano-grade wet grinder commercially available under the trade name “PUL-H/N” (manufactured and sold by Bühler AG, Uzwil, Switzerland). In a preferred embodiment of the present invention, a nano-grade wet grinder commercially available under the trade name “MiniCer” is used. The grinding balls have a diameter of about 0.05 mm to 1.0 mm. The grinding time is about 5 to about 300 minutes, preferably, about 30 to about 180 minutes. The speed is about 600 to about 4,000 rpm.

The centrifugal step of the process of the present invention can be carried out using any known centrifuges, for example, Beckman J2-MC Centrifuge manufactured in the U.S.A.

The stabilization effect of the aqueous dispersion containing nano/submicron, hydrophobic, functional compounds of the present invention is related to not only the grinding time, but also the amount of the stabilizer. The aqueous dispersion containing nano/submicron, hydrophobic, functional compounds of the present invention has improved bioavailability and is easily absorbed for use in cells and organism bodies to provide biological functions. For example, a nano/submicron curcumin solution providing anti-inflammatory effect in cell culture, promotes absorption within animal bodies up more than seven fold, and shows biological effect.

The following examples are illustrative and should not limit the scope of the present invention in any way. They demonstrate the aforementioned aspects and embodiments of the present invention in detail.

### EXAMPLES

### I. Design of Experiments

### 1. Preparation of a Complex Stabilizer

The present invention utilizes a complex stabilizer comprising lecithin and at least one non-phospholipid selected from polysorbate, sucrose ester, and polyglycerol fatty acid ester to allow hydrophobic, functional compounds to have good dispersibility in water.

In the following examples, the aqueous solution containing a complex stabilizer of the present invention is prepared by the following methods:
(A) Separately melting a predetermined amount of lecithin and a predetermined amount of non-phospholipid via heating; incorporating the non-phospholipid into the lecithin to formulate a complex stabilizer having a desired HLB value; incorporating the complex stabilizer into water in an amount of about 0.01% to about 10.0% (w/v) relative to the volume of water; and stirring the same under heating to form an aqueous solution containing a complex stabilizer; or
(B) Incorporating a predetermined amount of lecithin and a predetermined amount of non-phospholipid into water; homogenously stirring the same under heating to formulate an aqueous solution containing a complex stabilizer wherein the complex stabilizer in the aqueous solution containing a complex stabilizer has a desired HLB value.

### 2. Preparation of a Non-Homogenously Mixed Liquid Containing Hydrophobic, Functional Compounds

Hydrophobic, functional compounds, for example, CoQ10, lutene, silymarin, isoflavonoid, curcumin, etc., were incorporated into an aqueous solution containing a complex stabilizer prepared by method (A) or (B) mentioned above. After stirring, a non-homogenously mixed liquid was formed.

### 3. Preparation of an Aqueous Dispersion Containing Hydrophobic, Functional Compounds

The aforementioned non-homogenously mixed liquid was subjected to a homogenization pretreatment using a, homogenizer or an ultrasonic processor to form a homogenously mixed liquid. The homogenously mixed liquid was subjected to a nano-grade wet grinder commercially available under the trade name “MiniCer” (manufactured and sold by Netzsch-Feinmahltechnik GmbH, Selb, Germany). The speed was set at 1,500 rpm. The pressure was set at 4.5 bar. A frozen circulation tank was controlled at 7° C. An external double-layered cooling device was used to maintain the temperature of the liquid output from the milling chamber under 20° C. A peristaltic pump was used to control the flow speed at 400 to 800 mL/min. The homogenously mixed liquid was fed to the milling chamber with a filling ratio of 70% (v/v) of a milling media (yttria-stabilized tetragonal zirconia beads having a diameter of 0.05-1.0 mm) for milling. After milling, the liquid was passed through a sieve with meshes, which functions as a separating system for the milling media to control the particle size of the sample output from the milling chamber. Milling in this manner continued for 30 to 180 minutes. Sampling was done at a predetermined time for analysis. For a homogenization pretreatment carried using homogenizer, yttria-stabilized tetragonal zirconia beads having a diameter of 0.2 mm and 0.8 mm were used for milling. For a homogenization pretreatment carried using a ultrasonic processor, yttria-stabilized tetragonal zirconia beads having a diameter of 0.1 mm was used for milling. After milling, the particle size was distributed within nano and submicron ranges. A portion of the dispersion was transferred to a centrifuge (Beckman J2-MC Centrifuge manufactured in U.S.A.) for centrifugation at a speed of 12,000×g at 25° C. for 10 minutes. The supernatant was collected to obtain an aqueous dispersion containing a high concentration of nano/submicron, hydrophobic, functional compounds and having stable dispersibility. The aqueous dispersion containing a high concentration of nano, hydrophobic, functional compounds was subjected to particle size analysis and analysis of the concentration of the functional compounds, a test for determining anti-inflammatory activity on cells, an analysis for determining the concentration of the functional compounds in plasma after oral administration of rodents, and a test for determining anti-inflammatory activity in rodents.

### II. Analysis Assay

### A. Particle Size Analysis

1. Particle Size within the Range of 0.5-900 μm

A particle size analyzer “Mastersizer 2000” with Hydro 2000 Mu module (Malvern Instrument system Ltd, UK) was used for the particle size analysis. The parameter refraction index in water was set at 1.33. Sample unit selected was MS-14. The analysis mode was set to “polydisperse.” The active bean length was set at 2.4 mm. The speed of the pump was set at 2,000 rpm. The ultrasonic vibrating frequency was set at 10 kHz. After carrying out a background calibration of the device using deionized water at 25° C., a sample which was shaken for 3 minutes using an oscillator and degassed for 5 minutes using an ultrasonic processor (Branson 8210, Branson Ultrasonic Corp., Danbury, Conn., USA) was placed in the particle size analyzer. Analysis software was used to analyze scattering signals at a laser power of 70% or more and a covering rate within 10-30%. The number average particle diameter was calculated.

2. Particle Size within or Below the Range of 1.5-1,000 nm

The particle size analyzer PDDLS/BatchPlus System (Precision Detectors, Bellingham, Mass., USA) was used for particle size analysis. The parameter refraction index in water was set at 1.33. After carrying out a background calibration of the device using a sample having standard particle size (60 nm) at 25° C., a sample which was shaken for 3 minutes using an oscillator and degassed for 5 minutes using a ultrasonic processor (Branson 8210, Branson Ultrasonic Corp., Danbury, Conn., USA) was placed in the particle size analyzer. Analysis software was used to analyze scattering signals to obtain the number average particle diameter.

### B. Test for Determining Anti-Inflammatory Activity on Cells

1. Treatment of Cells

The method of Ĉiz et. al. (see reference 23) was incorporated herein for reference. A solution of RAW264.7 cells having a concentration of 1×10⁵ cells/well/100 μL was inoculated in a 96-well plate. The plate was placed into an incubator and incubated overnight (20-24 hours). Culture mediums contained lipopolysaccharide (LPS)(1 μg/mL) and samples were prepared at different concentrations (sample groups) or without any sample (positive control group). A culture medium without LPS and any sample (blank control group) was also prepared. Used culture medium was sucked out from the 96-well plate and 200 μL of newly prepared culture medium was injected into the plate, which was then placed back into the incubator for incubation for nitrogen oxide (NO) induction. After incubation overnight (16-20 hours), 100 μL of the supernatant was removed and placed into a new 96-well plate for NO determination. Another 96-well plate containing cells was prepared for determination of MTS cell viability.

2. Determination of the Amount of Nitrogen Oxide

The method of Kim et. al. (see reference 24) was incorporated herein for reference. 2.5% H₃PO₄ was used to prepare a 1% (w/v) solution of sulfanilamide and a 0.1% (w/v) solution of N-(1-naphthyl)ethyl-enediamine dihydrochloride. The two solutions were mixed at a ratio of 1:1 to produce a Griess reagent, which must not be exposed to light. Deionized water was used to the formulation of NaNO₂ solutions in a series of concentrations. These solutions were used as standard solutions. 100 μL of the cell supernatant or a standard solution was injected into a 96-hole plate. 100 μL of the Griess reagent was added. After the plate was kept away from being exposed to light for 5 minutes, absorbance at 540 nm was measured.

3. MTS Test of Cell Activity

A culture medium of cells was sucked out of the plate and the cells were washed once with a phosphate buffer solution (PBS). 100 μL of serum-free RPMI 1640 culture medium where MTS:RPMI=1:5 was placed into the plate. The plate was placed into an incubator for 10 minutes. Absorbance at 490 nm was measured.

4. Data Analysis

NO concentration was calculated from the absorbance of each sample on the basis of the calibration of concentration vs. absorbance measured using a NaNO₂ standard solution.

NO inhibition %=[1−(concentration of sample groups−concentration of blank group)/(concentration of control group−concentration of blank group)]×100/cell viability.

Cell viability was calculated using the absorbance measured by MTS.

Cell viability %=[(absorbance of sample groups−absorbance of blank group)/(absorbance of control group−absorbance of blank group)]×100.

### Example 1a

0.6 g of lecithin (HLB value: 8), 0.4 g of polysorbate 80 (HLB value: 15), and 0.2 g of sucrose stearate (HLB value: 15) (i.e., a total amount of 1.2 g (a concentration of 0.30% (w/v) relative to the volume of water)) were sequentially incorporated into 400 mL of water. The materials were homogenously stirred via heating to form an aqueous solution of a complex stabilizer having an HLB value of 11.5. 8 g of curcumin (a concentration of 2% (w/v) relative to the volume of water) was incorporated into the aqueous solution of a complex stabilizer. After stirring, a non-homogenously mixed liquid was obtained. The particle size of curcumin in the non-homogenously mixed liquid was measured. The non-homogenously mixed liquid was allowed to stand for 2 hours and then the particle size of curcumin in the non-homogenously mixed liquid was measured again. The result is shown in Table 1.

The non-homogenously mixed liquid was subjected to a homogenization pretreatment using a homogenizer (Pro-400 Pro Scientific Inc.; speed: 6,000 rpm; size of the head of the homogenizer: 10×150 mm; homogenization time: 10 minutes). After that, it was fed to a nano-grade wet grinder commercially available under the trade name “MiniCer” (manufactured and sold by Netzsch-Feinmahltechnik GmbH, Selb, Germany) in which the milling chamber was filled with 70% (v/v) of yttria-stabilized tetragonal zirconia beads with a diameter of 0.8 mm for circulation milling for 180 minutes. Sampling was made at the time after homogenization, and 30, 60, 150, and 180 minutes after milling. The sample was transferred to a centrifuge (Beckman J2-MC Centrifuge manufactured in U.S.A.) for centrifugation at a speed of 12,000×g at 25° C. for 10 minutes. The supernatant was collected. The Particle size and concentration of curcumin in the aqueous dispersion was measured. The result is shown in Table 1.

### Example 1b

0.6 g of lecithin (HLB value: 8), 0.4 g of polysorbate 80 (HLB value: 15), and 0.2 g of sucrose stearate (HLB value: 15) (i.e., a total amount of 1.2 g (a concentration of 0.30% (w/v) relative to the volume of water)) were sequentially incorporated into 400 mL of water. The materials were homogenously stirred via heating to form an aqueous solution of a complex stabilizer having an HLB value of 11.5. 8 g of curcumin (a concentration of 2% (w/v) relative to the volume of water) was incorporated into the aqueous solution of a complex stabilizer. After stirring, a non-homogenously mixed liquid was obtained. The particle size of curcumin in the non-homogenously mixed liquid was measured. The non-homogenously mixed liquid was allowed to stand for 2 hours and then the particle size of curcumin in the non-homogenously mixed liquid was measured again. The result is shown in Table 1.

The non-homogenously mixed liquid was subjected to a homogenization pretreatment using a homogenizer (Pro-400 Pro Scientific Inc.; speed: 6,000 rpm; size of the head of the homogenizer: 10×150 mm; homogenization time: 10 minutes). After that, it was fed to a nano-grade wet grinder commercially available under the trade name “MiniCer” (manufactured and sold by Netzsch-Feinmahltechnik GmbH, Selb, Germany) in which the milling chamber was filled with 70% (v/v) of yttria-stabilized tetragonal zirconia beads with a diameter of 0.2 mm for circulation milling for 180 minutes. Sampling was performed at the time after homogenization, and 30, 60, 150, and 180 minutes after milling. The sample was transferred to a centrifuge (Beckman 12-MC Centrifuge manufactured in U.S.A.) for centrifugation at a speed of 12,000×g at 25° C. for 10 minutes. The supernatant was collected. The particle size and concentration of curcumin in the aqueous dispersion was measured. The result is shown in Table 1.

### Example 1c

0.6 g of lecithin (HLB value: 8), 0.4 g of polysorbate 80 (HLB value: 15), and 0.2 g of sucrose stearate (HLB value: 15) (i.e., a total amount of 1.2 g (a concentration of 0.30% (w/v) relative to the volume of water)) were sequentially incorporated into 400 mL of water. The materials were homogenously stirred via heating to form an aqueous solution of a complex stabilizer having an HLB value of 11.5. 8 g of curcumin (a concentration of 2% (w/v) relative to the volume of water) was incorporated into the aqueous solution of a complex stabilizer. After stirring, a non-homogenously mixed liquid was obtained. The particle size of curcumin in the non-homogenously mixed liquid was measured. The non-homogenously mixed liquid was allowed to stand for 2 hours and then the particle size of curcumin in the non-homogenously mixed liquid was measured again. The result is shown in Table 1.

The non-homogenously mixed liquid was subjected to a homogenization pretreatment using an ultrasonic processor (Sonicator 4000 Ultrasonic Liquid Processors; the operating power and frequency are 600 W and 10 kHz respectively; a standard ½ inch diameter probe was used to treat the liquid for 15 minutes). After that, it was fed to a nano-grade wet grinder commercially available under the trade name “MiniCer” (manufactured and sold by Netzsch-Feinmahltechnik GmbH, Selb, Germany) in which the milling chamber was filled with 70% (v/v) of yttria-stabilized tetragonal zirconia beads with a diameter of 0.1 mm for circulation milling for 180 minutes. Sampling was performed at the time point after homogenization, and 30, 60, 150, and 180 minutes after milling. The sample was transferred to a centrifuge (Beckman J2-MC Centrifuge manufactured in U.S.A.) for centrifugation at a speed of 12,000×g at 25° C. for 10 minutes. The supernatant was collected. The particle size and concentration of curcumin in the aqueous dispersion was measured. The result is shown in Table 1.

TABLE 1

| | Example 1a | | | Example 1b | | | Example 1c | | |
|---|---|---|---|---|---|---|---|---|---|
| | Size of milling beads: 0.8 mm | | | Size of milling beads: 0.2 mm | | | Size of milling beads: 0.1 mm | | |
| | | Concen- | | | Concen- | | | Concen- | |
| | Particle | tration | | Particle | tration | | Particle | tration | |
| Milling time | size of | of nano- | Ratio of | size of | of nano- | Ratio of | size of | of nano- | Ratio of |
| (min) & after | curcumin | curcumin | nanoparticles | curcumin | curcumin | nanoparticles | curcumin | curcumin | nanoparticles |
| centrifugation | (nm) | (mg/ml) | (%) | (nm) | (mg/ml) | (%) | (nm) | (mg/ml) | (%) |
| Before | 4.229 ± 160 | 0.01 ± 0.01 | 0.05 ± 0.01 | 6.464 ± 309 | 0.01 ± 0.01 | 0.02 ± 0.01 | 5.321 ± 536 | 0.01 ± 0.01 | 0.04 ± 0.01 |
| homogenization | | | | | | | | | |
| After | 3.309 ± 125 | 0.03 ± 0.01 | 0.15 ± 0.01 | 4.189 ± 231 | 0.02 ± 0.01 | 0.10 ± 0.01 | 424 ± 52 | 0.13 ± 0.01 | 0.65 ± 0.01 |
| homogenization | | | | | | | | | |
| 30 | 133 ± 10 | 0.71 ± 0.02 | 0.85 ± 0.12 | 103 ± 25 | 1.58 ± 0.89 | 7.90 ± 1.05 | 74 ± 5 | 10.43 ± 3.24 | 52.15 ± 3.78 |
| 60 | 119 ± 8 | 0.93 ± 0.05 | 4.65 ± 0.58 | 95 ± 15 | 2.80 ± 0.58 | 14.00 ± 2.34 | 77 ± 3 | 12.41 ± 3.56 | 62.05 ± 2.55 |
| 150 | 89 ± 6 | 2.12 ± 0.35 | 10.60 ± 1.25 | 96 ± 8 | 8.14 ± 3.02 | 40.70 ± 4.25 | 81 ± 2 | 15.75 ± 4.02 | 78.75 ± 5.23 |
| 180 | 85 ± 5 | 2.23 ± 0.44 | 11.15 ± 1.32 | 91 ± 3 | 8.45 ± 2.43 | 42.25 ± 3.43 | 79 ± 1 | 15.26 ± 3.85 | 76.63 ± 5.27 |

Table 1 shows that after milling and centrifugation, the particle size of curcumin in the aqueous dispersion was reduced to nano-grade. Regarding the ratio of the nano-grade particles, the example in which yttria-stabilized tetragonal zirconia beads with a diameter of 0.1 mm were used achieves the highest-percentage (78.75%), the example in which yttria-stabilized tetragonal zirconia beads with a diameter of 0.2 mm were used achieves a second high percentage (42.25%), and the example in which yttria-stabilized tetragonal zirconia beads with a diameter of 0.1 mm were used achieves the lowest percentage (11.15%). In addition to achieving a higher percentage of nanoparticles, the example in which yttria-stabilized tetragonal zirconia beads with a smaller diameter were used achieves an enhanced milling efficacy. Moreover, table 1 shows that in the example using yttria-stabilized tetragonal zirconia beads with a diameter of 0.1 mm, the ratio of nanoparticles reaches 0.65% after vibration using an ultrasonic processor, which is 4-6 times the ratio achieved in the example in which a homogenizer (0.10-0.15%) was used. After vibration using a ultrasonic processor, the liquid was subjected a further milling for 30 minutes. At that time, the ratio of nanoparticles reached 52.15%, which is higher than the 42.25% achieved in the example using yttria-stabilized tetragonal zirconia beads with a diameter of 0.2 mm with milling carried out for 180 minutes. Given the above, it is clear that the milling time may be controlled within 30 minutes by using suitable homogenization means (for example, using an ultrasonic processor) and milling with yttria-stabilized tetragonal zirconia beads with a diameter of 0.1 mm.

If the species and ratio of a suitable stabilizer are not so selected, the chance that the particles of functional compounds collide with each other during milling will increase, which causes an increase of viscosity of the aqueous dispersion during milling, thereby limiting the diameter of yttria-stabilized tetragonal zirconia beads that can be used. Namely, larger yttria-stabilized tetragonal zirconia beads must be chosen. In this connection, Table 1 shows that an aqueous dispersion obtained by using larger yttria-stabilized tetragonal zirconia beads for milling had a ratio of nanoparticles of curcumin significantly lower than that of an aqueous dispersion obtained using smaller yttria-stabilized tetragonal zirconia beads.

According to the results of examples 1a, 1b, and 1c, using a complex stabilizer comprising lecithin and at least one non-phospholipid selected from polysorbate, sucrose ester, and polyglycerol fatty acid ester enables the use of yttria-stabilized tetragonal zirconia beads with a small diameter of 0.1 mm for milling, enhances milling efficacy, and significantly increases the ratio of nanoparticles of curcumin.

### Example 2

### I. Influence of Lecithin on the Concentration of an Aqueous Dispersion Containing Nano-Curcumin

0.4 g (a concentration of 0.1% (w/v) relative to the volume of water), 0.8 g (a concentration of 0.2% (w/v) relative to the volume of water), 1.2 g (a concentration of 0.3% (w/v) relative to the volume of water), 1.6 g (a concentration of 0.4% (w/v) relative to the volume of water), and 2.0 g (a concentration of 0.5% (w/v) relative to the volume of water) of lecithin were weighed respectively and incorporated into 400 mL of water. The materials were homogenously stirred via heating. 4 g of curcumin (a concentration of 1% (w/v) relative to the volume of water) was incorporated into the aforementioned liquid. After stirring, a non-homogenously mixed liquid was obtained. The non-homogenously mixed liquid was allowed to stand for 2 hours and then the concentration of curcumin in the non-homogenously mixed liquid was measured. The result is shown in Table 2A.

The non-homogenously mixed liquid was subjected to a homogenization pretreatment using a homogenizer (Pro-400 Pro Scientific Inc.; speed: 6,000 rpm; size of the head of the homogenizer: 10×150 mm; homogenization time: 10 minutes). After that, it was fed to a nano-grade wet grinder commercially available under the trade name “MiniCer” (manufactured and sold by Netzsch-Feinmahltechnik GmbH, Selb, Germany) in which the milling chamber was filled with 70% (v/v) of yttria-stabilized tetragonal zirconia beads with a diameter of 0.2 mm for circulation milling for 180 minutes. After milling, the aqueous dispersion was transferred to a centrifuge (Beckman J2-MC Centrifuge manufactured in U.S.A.) for centrifugation at a speed of 12,000×g at 25° C. for 10 minutes. The supernatant was collected to obtain an aqueous dispersion containing nano-curcumin. The concentration of curcumin in the aqueous dispersion was measured again. The result is shown in Table 2A.

In all examples, the concentration of the nano/submicron curcumin was measured. Except for the example in which lecithin was not added (in which the concentration is only about 0.1 mg/mL), the rest of the examples in which curcumin was added at different concentrations showed a concentration of curcumin of about 10 mg/mL. Table 2A shows that incorporation of lecithin enhances the concentration of a liquid containing nano-curcumin compared to a similar liquid without lecithin. The example in which lecithin was added in an amount of 0.2% (w/v) achieved the highest concentration, 1.79 mg/mL, and the ratio of nanoparticales was 17.9%. However, it is still lower than the concentration achieved by using a complex stabilizer (i.e., 20% or higher) (see Table 2B). ROC (Taiwan) patent publication no. 200533387 (see reference 16) discloses the preparation of a drug-phospholipid complex using a phospholipid and a drug via nano-grade wet grinding. According to the result of example 2A, it is clear that although a phospholipid may increase the dispersibility of a hydrophobic substance, the level of increase is quite limited. For a nano/submicron curcumin dispersion in which a phospholipid was incorporated as a single stabilizer, the level of increase of the nanoparticles seems to be limited.

TABLE 2A

| | Concentration of nano-curcumin (mg/mL) | | | | | |
|---|---|---|---|---|---|---|
| | Native | 0.1% L | 0.2% L | 0.3% L | 0.4% L | 0.5% L |
| Before | 0 ± 0.00 | 0 ± 0.00 | 0 ± 0.00 | 0 ± 0.00 | 0 ± 0.00 | 0 ± 0.00 |
| homogenization | | | | | | |
| After grinding and | 0.09 ± 0.01 | 1.13 ± 0.55 | 1.79 ± 0.86 | 1.47 ± 0.43 | 0.33 ± 0.01 | 0.33 ± 0.01 |
| centrifuging* | | | | | | |
| Ratio of | — | 11.3 | 17.9 | 14.7 | 3.3 | 3.3 |
| nanoparticles (%) | | | | | | |

Native: without adding any stabilizer.
L: lecithin
*A supernatant obtained after centrifugation at a speed of 12,000 xg.

### II. Influence of Complex Stabilizers Comprising Different Combinations on the Concentration of an Aqueous Dispersion Containing Nano-Curcumin

The following stabilizers were used in these examples:
Stabilizer (1): 0.4 g (a concentration of 0.1% (w/v) relative to the volume of water) of polysorbate 20 (HLB value: 16.7) was incorporated;
Stabilizer (2): 0.4 g (a concentration of 0.1% (w/v) relative to the volume of water) of sucrose stearate (HLB value: 15) was incorporated;
Stabilizer (3): 0.4 g of lecithin (HLB value: 8) and 0.2 g of polysorbate 20 (HLB value: 16.7) were weighed (i.e., a total combined weight 0.6 g (a concentration of 0.15% (w/v) relative to the volume of water)) and melted by heating. After that, polysorbate 20 was gradually incorporated into the lecithin and the resulting melt was stirred homogenously to formulate a complex stabilizer having HLB value of 10.5;
Stabilizer (4): 0.5 g of lecithin (HLB value: 8) and 0.3 g of sucrose stearate (HLB value: 15) were weighed (i.e., a total weight 0.8 g (a concentration of 0:20% (w/v) relative to the volume of water)) and melted by heating. After that, sucrose stearate was gradually incorporated into the lecithin and the resulting melt was stirred homogenously to formulate a complex stabilizer having HLB value of 10.5:
Stabilizer (5): 0.1 g of lecithin (HLB value: 8) and 0.5 g of palmatic acid sucrose ester (HLB value: 11) were weighed (i.e., a total combined weight 0.6 g (a concentration of 0.15% (w/v) relative to the volume of water)) and melted by heating. After that, palmatic acid sucrose ester was gradually incorporated into the lecithin and the resulting melt was stirred homogenously to formulate a complex stabilizer having HLB value of 10.5;
Stabilizer (6): 0.4 g of lecithin (HLB value: 8). 0.1 g of stearic acid sucrose ester (HLB value: 15), and 0.1 g of polysorbate 80 (HLB value: 15) were weighed (i.e., a total combined weight 0.6 g (a concentration of 0.15% (w/v) relative to the volume of water)) and melted by heating. After that, sucrose stearate and polysorbate 80 were incorporated into the lecithin and the resulting melt was stirred homogenously to formulate a complex stabilizer having HLB value of 10.5;
Stabilizer (7): 0.2 g of lecithin (HLB value: 4) and 0.4 g of polycerol stearate (HLB value: 14) were weighed (i.e., a total combined weight 0.6 g (a concentration of 0.15% (w/v) relative to the volume of water)) and melted by heating. After that, polycerol stearate was incorporated into the lecithin and the resulting melt was stirred homogenously to formulate a complex stabilizer having HLB value of 10.5;
Stabilizer (8): 0.34 g of lecithin (HLB value: 8). 0.17 g of stearic acid sucrose ester (HLB value: 15), and 0.17 g of polycerol stearate (HLB value: 11) were weighed (i.e., a total combined weight 0.68 g (a concentration of 0.17% (w/v) relative to the volume of water)) and melted by heating. After that, sucrose stearate and polycerol stearate were gradually incorporated into the lecithin and the resulting melt was stirred homogenously to formulate a complex stabilizer having HLB value of 10.5.

The aforementioned stabilizers were incorporated into 400 mL of water respectively, and stirred homogenously under heating. 4 g of curcumin (a concentration of 1% (w/v) relative to the volume of water) were incorporated into each solution. After stirring, a non-homogenously mixed liquid was formed. The non-homogenously mixed liquid was allowed to stand for 2 hours. The concentration of curcumin of each liquid was measured. The result is shown in Table 2B.

These non-homogenously mixed liquids were subjected to a homogenization pretreatment using a homogenizer (Pro-400 Pro Scientific Inc.; speed: 6,000 rpm; size of the head of the homogenizer: 10×150 mm; homogenization time: 10 minutes). After that, they were fed to a nano-grade wet grinder commercially available under the trade name “MiniCer” (manufactured and sold by Netzsch-Feinmahltechnik GmbH, Selb, Germany) in which the milling chamber was filled with 70% (v/v) of yttria-stabilized tetragonal zirconia beads with a diameter of 0.2 mm for circulation milling for 180 minutes. The dispersions were allowed to stand for 2 hours. The concentration of curcumin of each dispersion was measured. After milling, the dispersions were transferred to a centrifuge (Beckman J2-MC Centrifuge manufactured in U.S.A.) for centrifugation at a speed of 12,000×g at 25° C. for 10 minutes. The supernatant was collected to obtain an aqueous dispersion containing nano-curcumin. At this time point, the concentration of curcumin of each dispersion was measured again. The result is shown in Table 2B.

In the above examples in which various stabilizers were added, the concentration of nano/submicron curcumin after wet grinding was about 10 mg/mL, which is close to the operating concentration (the initial concentration relative to the volume of water). The result of Table 2B shows that using a non-phospholipid as the single stabilizer, for example, Polysorbate 80 (stabilizer (1)) and sucrose stearate (stabilizer (2)) can only slightly increase the concentration of curcumin dispersed in the solution. Their concentrations are 0.24 mg/mL and 0.45 mg/mL, respectively (note: The concentration of the solution without any stabilizer is only 0.09 mg/mL (see Table 2A)) and their nanoparticles are 2.4% and 4.5% on the basis of the total particles, respectively. This efficacy is clearly inferior to that achieved by the example in which lecithin was used as the single stabilizer (see Table 2A). Using a complex stabilizer (stabilizers (3)-(8)) of lecithin and at least one non-phospholipid significantly increases the concentration of the nano-curcumin dispersion. The concentration ranges from 2.22 to 4.61 mg/mL and the nanoparticles are in the range from 22.2% to 46.1% on the basis of the total particles (see Table 2B). This efficacy is higher than that achieved by the example in which lecithin was used as the single stabilizer (see Table 2A), and also higher than that achieved by the examples in which a non-phospholipid (polysorbate or sucrose stearate) was used as the single stabilizer (see Table 2A, stabilizers (1) and (2)). Therefore, using a complex stabilizer can significantly increase the ratio of nano-curcumin.

TABLE 2B

| | Concentration of nano-curcumin (mg/mL) | | | | | | | |
|---|---|---|---|---|---|---|---|---|
| | Stabilizer | Stabilizer | Stabilizer | Stabilizer | Stabilizer | Stabilizer | Stabilizer | Stabilizer |
| | (1) | (2) | (3) | (4) | (5) | (6) | (7) | (8) |
| Before | 0 ± 0.00 | 0 ± 0.00 | 0 ± 0.00 | 0 ± 0.00 | 0.01 ± 0.00 | 0.01 ± 0.00 | 0.01 ± 0.00 | 0.02 ± 0.00 |
| homogenization | | | | | | | | |
| After milling | 0.24 ± 0.01 | 0.45 ± 0.01 | 2.84 ± 0.43 | 3.23 ± 0.36 | 3.70 ± 0.24 | 2.30 ± 0.14 | 2.22 ± 0.14 | 4.61 ± 0.38 |
| and | | | | | | | | |
| centrifuging* | | | | | | | | |
| Ratio of | 2.4 | 4.5 | 28.4 | 32.3 | 37.0 | 23.0 | 22.2 | 46.1 |
| nanoparticles | | | | | | | | |
| (%) | | | | | | | | |

*A supernatant obtained after centrifugation at a speed of 12,000 xg.

### III. Influence of the Ratio of Curcumin to a Complex Stabilizer and Grinding Time on the Concentration of an Aqueous Dispersion Containing Nano-Curcumin

In this example, an aqueous solution of a complex stabilizer comprising lecithin and polysorbate 20 and having HLB value of 10.5 was prepared. The amount of the complex stabilizer relative to the volume of water was selected as 0.1% (w/v). 0.15% (w/v), and 0.3% (w/v). The following methods were used for the preparation:
(1) For an aqueous solution containing a complex stabilizer in an amount of 0.1% relative to the volume of water: 0.28 g of lecithin (HLB value: 8) and 0.12 g of polysorbate 20 (HLB value: 16.7) were sequentially incorporated into 400 mL of water. After stirring homogenously under heating, an aqueous solution containing a complex stabilizer having an HLB value of 10.5 was prepared.
(2) For an aqueous solution containing a complex stabilizer in an amount of 0.15% relative to the volume of water: 0.43 g of lecithin (HLB value: 8) and 0.17 g of polysorbate 20 (HLB value: 16.7) were sequentially incorporated into 400 mL of water. After stirring homogenously under heating, an aqueous solution containing a complex stabilizer having an HLB value of 10.5 was prepared.
(3) For an aqueous solution containing a complex stabilizer in an amount of 0.30% relative to the volume of water: 0.85 g of lecithin (HLB value: 8) and 0.35 g of polysorbate 20 (HLB value: 16.7) were sequentially incorporated into 400 mL of water. After stirring homogenously under heating, an aqueous solution containing a complex stabilizer having an HLB value of 10.5 was prepared.

4 g of curcumin (a concentration of 1% (w/v) relative to the volume of water) was incorporated into each of the aforementioned aqueous solutions containing a complex stabilizer. After stirring, a non-homogenously mixed liquid was formed. The particle size of the non-homogenously mixed liquids was measured. The weight ratio of curcumin to the complex stabilizer was 10:1 (i.e., the solution comprises 0.1% of the stabilizer), 6.67:1 (i.e., the solution comprises 0.15% of the stabilizer), and 3.33:1 (i.e., the solution comprises 0.3% of the stabilizer). The non-homogenously mixed liquids were allowed to stand for 2 hours. The concentration of curcumin of each liquid was measured. The result is shown in Table 2C.

The non-homogenously mixed liquids were subjected to a homogenization pretreatment using a homogenizer (Pro-400 Pro Scientific Inc.; speed: 6,000 rpm; size of the head of the homogenizer: 10×150 mm; homogenization time: 10 minutes). After that, they were fed to a nano-grade wet grinder commercially available under the trade name “MiniCer” (manufactured and sold by Netzsch-Feinmahltechnik GmbH, Selb, Germany) in which the milling chamber was filled with 70% (v/v) of yttria-stabilized tetragonal zirconia heads with a diameter of 0.2 mm for circulation milling for 180 minutes. The dispersion was allowed to stand for 2 hours and then concentration of curcumin in the non-homogenously mixed liquid was measured. After milling, the aqueous dispersion was transferred to a centrifuge (Beckman J2-MC Centrifuge manufactured in U.S.A.) for centrifugation at a speed of 12,000×g at 25° C. for 10 minutes. The supernatant was collected to obtain an aqueous dispersion containing nano-curcumin. The concentration of curcumin in each aqueous dispersion was measured again. The result is shown in Table 2C.

Table 2C shows that for the solution containing the complex stabilizer in an amount of 0.1% (the weight ratio of curcumin to the complex stabilizer in the solution is 10:1), 0.15% (the weight ratio of curcumin to the complex stabilizer in the solution is 6.67:1), and 0.3% (the weight ratio of curcumin to the complex stabilizer in the solution is 3.33:1), the concentrations of the aqueous dispersions containing nano-curcumin are 1.18, 2.84, and 1.96 mg/mL, respectively, after being milled for 180 minutes. Clearly, the weight ratio of curcumin to the complex stabilizer in the solution has an influence on the concentration of the resulting aqueous dispersion containing nano-curcumin. In addition, the milling time also has an influence on the concentration and particle size of the resulting aqueous dispersion containing nano-curcumin. For a solution containing a complex stabilizer in an amount of 0.1%, an aqueous dispersion of nano-curcumin with the highest concentration, 1.34 mg/mL, was obtained after milling for 120 minutes. After milling for 180 minutes, the smallest particle size, 88 nm, was achieved. For solutions containing a complex stabilizer in an amount of 0.15% and 0.3%, aqueous dispersions of nano-curcumin with their highest concentrations were obtained after milling for 180 minutes, which were 2.84 and 1.96 mg/mL, respectively. Their smallest particle sizes were 97 nm and 94 nm, respectively. Given the above, it is clear that the stabilization efficacy is not only related to the weight ratio (the optimized ratio) of curcumin to the complex stabilizer, but also the length of milling time.

TABLE 2C

| | Amount of a complex stabilizer in solution | | | | | |
|---|---|---|---|---|---|---|
| Milling time | 0.1% (w/v) | | 0.15% (w/v) | | 0.3% (w/v) | |
| (min) & | Concentration | | Concentration | | Concentration | |
| after | of the dispersion | Particle size | of the dispersion | Particle size | of the dispersion | Particle size |
| centrifugation* | (mg/ml) | (nm) | (mg/ml) | (nm) | (mg/ml) | (nm) |
| Before | 0.01 ± 0.00 | 5.705 ± 578 | 0.02 ± 0.00 | 8.087 ± 860 | 0.03 ± 0.00 | 5.140 ± 178 |
| homogenization | | | | | | |
| 30 | 0.56 ± 0.08 | 241 ± 21 | 0.62 ± 0.03 | 174 ± 60 | 0.14 ± 0.09 | 146 ± 12 |
| 120 | 1.34 ± 0.15 | 102 ± 3 | 2.11 ± 0.18 | 101 ± 3 | 1.46 ± 0.14 | 117 ± 6 |
| 180 | 1.18 ± 0.18 | 88 ± 8 | 2.84 ± 0.43 | 97 ± 7 | 1.96 ± 0.12 | 94 ± 12 |

*A supernatant obtained after centrifugation at a speed of 12,000 xg.

### IV. Proportion of Nano-Grade Particles on the Basis of Nano/Submicron Particles

(1) Preparation of an Aqueous Dispersion Comprising Nano/Submicron Curcumin with a Concentration Close to 30 mg/mL

2.5 g of lecithin (HLB value: 10) and 1.1 g of polysorbate 20 (HLB value: 16.7) were sequentially incorporated into 400 mL of water. The total weight was 3.6 g (a concentration of 0.90% (w/v) relative to the volume of water). After stirring homogenously under heating, an aqueous solution containing a complex stabilizer having an HLB value of 12 was prepared. 12 g of curcumin (a concentration of 3% (w/v) relative to the volume of water) was incorporated into the aqueous solution containing a complex stabilizer. After stirring, a non-homogenously mixed liquid was formed. A small portion of the non-homogenously mixed liquids was allowed to stand for 2 hours. The concentration of curcumin in the solution was measured. The result is shown in Table 2D. Another small portion of the non-homogenously mixed liquids was subjected to a homogenization pretreatment using a homogenizer (Pro-400 Pro Scientific Inc.; speed: 6,000 rpm; size of the head of the homogenizer: 10×150 mm; homogenization time: 10 minutes). The supernatant was collected. The particle size and concentration of curcumin were measured again. The result is shown in Table 2D.

After that, the aforementioned non-homogenously mixed liquids were subjected to a homogenization pretreatment using a homogenizer (Pro-400 Pro Scientific Inc.; speed: 6,000 rpm; size of the head of the homogenizer: 10×150 mm; homogenization time: 10 minutes), and then fed to a nano-grade wet grinder commercially available under the trade name “MiniCer” (manufactured and sold by Netzsch-Feinmahltechnik GmbH, Selb, Germany) in which the milling chamber was filled with 70% (v/v) of yttria-stabilized tetragonal zirconia beads with a diameter of 0.2 mm for circulation milling for 180 minutes. The particle size of the particles in the dispersion was measured. The dispersion was allowed to stand for 2 hours and the concentration of curcumin was measured. The result is shown in Table 2D. After milling, the aqueous dispersion was transferred to a centrifuge (Beckman J2-MC Centrifuge manufactured in U.S.A.) for centrifugation at a speed of 12,000×g at 25° C. for 10 minutes. The supernatant was collected to obtain an aqueous dispersion containing nano-curcumin. The concentration of curcumin in each aqueous dispersion was measured again. The result is shown in Table 2D.

Table 2D shows that before homogenization, the non-homogenously mixed liquid had a particle size of 10,970 nm and a concentration of 0.18 mg/mL. After centrifugation, the particle size became 3,725 nm and the concentration became 0.08 mg/mL. Clearly, the non-homogenously mixed liquid had a relatively large particle size and a low concentration of curcumin. After incorporating a complex stabilizer into the dispersion and subjecting it to a nano-grade wet grinding miller for milling, the particle size of curcumin was 285 nm, which is within the range of nanometer scale for food sector. The dispersion retained good dispersibility after standing. The concentration of nano/submicron in the dispersion was 29.7 mg/mL, which is close to the operating concentration (the initial concentration relative to the volume of water) of about 30 mg/mL. Therefore, almost all of the incorporated components were formulated into the nano/submicron dispersion. When the dispersion was subjected to a centrifugation step, a nano-grade dispersion which has a stable dispersibility, a particle size of 81 nm, and a concentration of 25.26 mg/mL was obtained. This shows that after milling, the aqueous dispersion comprised a combination of nano particles and submicron particles. After centrifugation, the nanoparticles comprised 85% of the total particles.

(2) Preparation of an Aqueous Dispersion Comprising Nano/Submicron Curcumin with a Concentration Over 100 mg/mL

10 g of lecithin (HLB value: 8), 4.5 g of polysorbate 20 (HLB value: 16:7), and 3.5 g sucrose stearate (HLB value: 15) (i.e., a total weight of 18 g (a concentration of 4.5% (w/v) relative to the volume of water)) were sequentially incorporated into 400 mL of water. After stirring homogenously under heating, an aqueous solution containing a complex stabilizer having an HLB value of 11.5 was prepared. 60 g of curcumin (a concentration of 15% (w/v) relative to the volume of water) were incorporated into the aqueous solution containing a complex stabilizer. After stirring, a non-homogenously mixed liquid was formed. The particle size of curcumin in the mixed liquid was measured. A small portion of the non-homogenously mixed liquid was allowed to stand for 2 hours and the concentration of curcumin in the mixed liquid was measured. The result is shown in Table 2E. Another small portion of the non-homogenously mixed liquids was subjected to a homogenization pretreatment using a homogenizer (Pro-400 Pro Scientific Inc.; speed: 6,000 rpm; size of the head of the homogenizer: 10×150 mm; homogenization time: 10 minutes). The supernatant was collected. The particle size and concentration of curcumin were measured again. The result is shown in Table 2E.

The aforementioned non-homogenously mixed liquids were subjected to a homogenization pretreatment using a homogenizer (Pro-400 Pro Scientific Inc.; speed: 6,000 rpm; size of the head of the homogenizer: 10×150 mm; homogenization time: 10 minutes), and then fed to a nano-grade wet grinder commercially available under the trade name “MiniCer” (manufactured and sold by Netzsch-Feinmahltechnik GmbH, Selb, Germany) in which the milling chamber was filled with 70% (v/v) of yttria-stabilized tetragonal zirconia beads with a diameter of 0.2 mm for circulation milling for 180 minutes. The particle size of the particles in the dispersion was measured. The dispersion was allowed to stand for 2 hours and the concentration of curcumin was measured. The result is shown in Table 2E. After milling, the aqueous dispersion was transferred to a centrifuge (Beckman 12-MC Centrifuge manufactured in U.S.A.) for centrifugation at a speed of 12,000×g at 25° C. for 10 minutes. The supernatant was collected to obtain an aqueous dispersion containing nano-curcumin. The concentration of curcumin in each aqueous dispersion was measured again. The result is shown in Table 2E.

Table 2E shows that before homogenization, the non-homogenously mixed liquid had a particle size of 12,221 nm and a concentration of 0.14 mg/mL. After centrifugation, the particle size became 3,421 nm and the concentration became 0.06 mg/mL. Clearly, the non-homogenously mixed liquid had a relatively large particle size and a low concentration of curcumin. After incorporating a complex stabilizer into the dispersion and subjecting it to a nano-grade wet grinding miller for milling, the particle size of curcumin was 310 nm, which is within the range of submicron particles. The dispersion retained good dispersibility after standing. The concentration of nano/submicron in the dispersion was 130.10 mg/mL, which is close to the operating concentration (the initial concentration relative to the volume of water; for example, if 60 g curcumin was incorporated into 400 mL of water, presuming that curcumin has a volume close to that of water, the total volume was 460 mL) of about 130 mg/mL. Therefore, almost all of the incorporated components were formulated into the nano/submicron dispersion. If the dispersion was subjected to a centrifugation step, a nano-grade dispersion which has a stable dispersibility, a particle size of 147 nm, and a concentration of 97.25 mg/mL may be obtained. It shows that after milling, the aqueous dispersion comprised a combination of nano particles and submicron particles. After centrifugation, the nanoparticles comprised 75% of the total particles.

According to the results of Tables 2D and 2E, it is clear that before homogenization, the non-homogenously mixed liquid had a particle size significantly larger than or close to 10,000 nm. After standing for 2 hours, a majority of the particles precipitated. Therefore, the dispersion had a low concentration of curcumin. If a complex stabilizer was incorporated into the dispersion and the dispersion was subjected to a nano-grade wet grinding miller for milling, curcumin had a particle size significantly smaller than 1,000 nm, which is within the range of submicron particles. After standing, the dispersions retained a good dispersibility and a significantly high concentration (i.e. close to the initial concentration) of curcumin. After being subjected to centrifugation at a speed of 12,000×g, a nano-grade dispersion having stable dispersibility and a particle size smaller than or close to 100 nm was obtained. The two examples respectively show that the nanoparticles comprised 75% and 85% of the total particles, each of which represents a substantially high ratio.

TABLE 2D

| | Concentration of | | | Particle size of | |
|---|---|---|---|---|---|
| | curcumin (mg/mL) | | | curcumin (nm) | |
| | Mixed liquid or | After | Ratio of | Mixed | Post |
| | dispersion | centrifugation | nano-grade | liquid or | centrifugation |
| Treatment | Stand for 2 hrs | supernatant** | particles (%) | dispersion* | supernatant** |
| Before | 0.18 ± 0.01 | 0.08 ± 0.01 | 0 | 10,970 ± 106 | 3,725 ± 450 |
| homogenization | | | | | |
| After milling | 29.70 ± 0.75 | 25.26 ± 0.92 | 85 ± 5 | 285 ± 6 | 81 ± 18 |

*Particle size was measured immediately after stirring or milling following incorporation of curcumin.
**A supernatant obtained after centrifugation at a speed of 12,000 xg.

TABLE 2E

| | Concentration of | | | Particle size of | |
|---|---|---|---|---|---|
| | curcumin (mg/mL) | | | curcumin (nm) | |
| | Mixed liquid or | After | Ratio of | Mixed | Post |
| | dispersion | centrifugation | nano-grade | liquid or | centrifugation |
| Treatment | Stand for 2 hrs | supernatant** | particles (%) | dispersion* | supernatant** |
| Before | 0.14 ± 0.01 | 0.06 ± 0.01 | 0 | 12,221 ± 566 | 3,421 ± 247 |
| homogenization | | | | | |
| After milling | 130.10 ± 4.23 | 97.25 ± 4.46 | 75 ± 4 | 310 ± 15 | 147 ± 20 |

*Particle size was measured immediately after stirring or milling following incorporation of curcumin.
**A supernatant obtained after centrifugation at a speed of 12,000 xg.

### Example 3

1.5 g of lecithin (HLB value: 8), 1.1 g of polysorbate 20 (HLB value: 16.7), and 1.0 g of sucrose stearate (HLB value: 15) (i.e., a total amount of 3.6 g (a concentration of 0.9% (w/v) relative to the volume of water)) were melted under heating separately. Polysorbate 20 and sucrose stearate were sequentially incorporated into lecithin. The materials were homogenously stirred to form a complex stabilizer having an HLB value of 12.5. The complex stabilizer was incorporated into 400 mL of water. The materials were homogenously stirred under heating. 12 g (a concentration of 3% (w/v) relative to the volume of water) of CoQ10, or lutene, sesamin, silymarin, isoflavonoid, or hesperidin was incorporated into the aqueous solution of a complex stabilizer. After stirring, a non-homogenously mixed liquid was obtained. The particle size of the non-homogenously mixed liquid was measured. A small portion of the non-homogenously mixed liquid was allowed to stand for 2 hours and then the concentration of the functional compounds in the solution was measured. The result is shown in Tables 3A and 3B. Another portion of the non-homogenously mixed liquid comprising silymarin, lutene, or isoflavonoid was subjected to testing for anti-inflammatory activity on cells as described in Example 6. A further another portion of the non-homogenously mixed liquid was subjected to a centrifugation step at a speed of 12,000×g at 25° C. for 10 minutes. The supernatant was collected. The particle size and concentration of the dispersion were measured. The result is shown in Tables 3A and 3B.

The aforementioned non-homogenously mixed liquids were subjected to a homogenization pretreatment using a homogenizer (Pro-400 Pro Scientific Inc.; speed: 6,000 rpm; size of the head of the homogenizer: 10×150 mm; homogenization time: 10 minutes), and then fed to a nano-grade wet grinder commercially available under the trade name “MiniCer” (manufactured and sold by Netzsch-Feinmahltechnik GmbH. Selb, Germany) in which the milling chamber was filled with 70% (v/v) of yttria-stabilized tetragonal zirconia beads with a diameter of 0.2 mm for circulation milling for 180 minutes. The particle size of the particles in the dispersion was measured. The dispersion was allowed to stand for 2 hours and the concentration of the functional material was measured. The result is shown in Tables 3A and 3B. Moreover, dispersions comprising silymarin, lutene, and isoflavonoid obtained after milling were subjected to testing for anti-inflammatory activity on cells as described in Example 6. After milling, the aqueous dispersion was transferred to a centrifuge (Beckman J2-MC Centrifuge manufactured in U.S.A.) for centrifugation at a speed of 12,000×g at 25° C. for 10 minutes. The supernatant was collected to obtain an aqueous dispersion containing nano-grade functional compounds. The concentration and particle size of curcumin in the aqueous dispersion was measured again. The result is shown in Tables 3A and 3B. The dispersions comprising silymarin, lutene, and isoflavonoid obtained after centrifugation were subjected to testing for anti-inflammatory activity on cells as described in Example 6.

Tables 3A and 3B show that before homogenization, except for the lutene and isoflavonoid with particle size ranging from 6,000-7,000 nm, the rest of the functional compounds within the non-homogenously mixed liquids had particle size larger or significantly larger than 10,000 nm. After centrifugation, particle size was approximately 5,000 nm. Clearly, the particle size in these non-homogenously mixed liquids was relatively large. If a complex stabilizer was incorporated into the dispersions and the dispersions were subjected to a nano-grade wet grinding miller for milling, the particles had a particle size of 200-1,600 nm, which is within the range of submicron particles. Depending on the species of the functional compounds, after standing, the dispersions retained good dispersibility. After being subjected to centrifugation at a speed of 12,000×g, a nano-grade dispersion having stable dispersibility and a particle size of about 20-150 nm was obtained. This shows that after milling, the aqueous dispersion comprised a combination of nano particles and submicron particles. The nanoparticles comprised 42-76% of the total particles. Therefore, the preparation method of the present invention effectively enhanced the disperibility of the aforementioned hydrophobic functional compounds.

TABLE 3A

| | CoQ10 | | | | lutene | |
|---|---|---|---|---|---|---|
| | Particle size (nm) | | Concentration (mg/mL) | | Particle size (nm) | |
| | | Supernatant | Dispersion, | Supernatant | | Supernatant |
| | | obtained after | Standing for | obtained after | | obtained after |
| Treatment | Dispersion | centrifugation** | 2 hrs | centrifugation** | Dispersion | centrifugation** |
| Before | 28.215 ± 1.040 | 4.423 ± 264 | 1.9 ± 0.89 | 0.7 ± 0.10 | 6.017 ± 513 | 5.550 ± 457 |
| homogenization | | | | | | |
| After milling* | 1.308 ± 85 | 151 ± 10 | 19.1 ± 0.42 | 8.9 ± 0.15 | 650 ± 30 | 20 ± 2 |

| | |
|---|---|
| Ratio of | 46.60 |
| nano-grade | |
| particles (%) | |

| | lutene | | sesamin | | | |
|---|---|---|---|---|---|---|
| | Concentration (mg/mL) | | Particle size (nm) | | Concentration (mg/mL) | |
| | Dispersion, | Supernatant | | Supernatant | Dispersion, | Supernatant |
| | Standing for | obtained after | | obtained after | standing | obtained after |
| Treatment | 2 hrs | centrifugation** | Dispersion | centrifugation** | for 2 hrs | centrifugation** |
| Before | 1.2 ± 1.01 | 0.7 ± 0.53 | 14.485 ± 984 | 5.394 ± 352 | 0.9 ± 0.95 | 0.7 ± 0.45 |
| homogenization | | | | | | |
| After milling* | 23.1 ± 0.66 | 14.9 ± 0.40 | 1.658 ± 94 | 123 ± 15 | 13.0 ± 1.03 | 8.8 ± 0.36 |

| | | |
|---|---|---|
| Ratio of | 64.50 | 67.69 |
| nano-grade | | |
| particles (%) | | |

*Homogenization and milling using yttria-stabilized tetragonal zirconia beads having a diameter of 0.2 mm for 180 minutes.
**Nano-grade supernatant obtained after milling and centrifugation at a speed of 12,000 xg.

TABLE 3B

| | CoQ10 | | | | lutene | |
|---|---|---|---|---|---|---|
| | Particle size (nm) | | Concentration (mg/mL) | | Particle size (nm) | |
| | | Supernatant | Dispersion, | Supernatant | | Supernatant |
| | | obtained after | Standing for | obtained after | | obtained after |
| Treatment | Dispersion | centrifugation** | 2 hrs | centrifugation** | Dispersion | centrifugation** |
| Before | 9.5610 ± 6.425 | 6.670 ± 853 | 1.0 ± 0.05 | 0.8 ± 0.47 | 7.418 ± 499 | 5.387 ± 397 |
| homogenization | | | | | | |
| After milling* | 96 ± 5 | 30 ± 10 | 22.7 ± 5.73 | 9.6 ± 0.15 | 245 ± 40 | 98 ± 8 |

| | |
|---|---|
| Ratio of | 42.29 |
| nano-grade | |
| particles (%) | |

| | lutene | | sesamin | | | |
|---|---|---|---|---|---|---|
| | Concentration (mg/mL) | | Particle size (nm) | | Concentration (mg/mL) | |
| | Dispersion, | Supernatant | | Supernatant | Dispersion, | Supernatant |
| | Standing for | obtained after | | obtained after | standing for | obtained after |
| Treatment | 2 hrs | centrifugation** | Dispersion | centrifugation** | 2 hrs | centrifugation** |
| Before | 1.1 ± 1.52 | 0.8 ± 0.19 | 22.277 ± 394 | 4.375 ± 135 | 1.0 ± 0.61 | 0.6 ± 0.94 |
| homogenization | | | | | | |
| After milling* | 19.4 ± 0.24 | 14.7 ± 0.45 | 443 ± 21 | 123 ± 9 | 21.6 ± 2.06 | 9.6 ± 0.47 |

| | | |
|---|---|---|
| Ratio of | 75.77 | 44.44 |
| nano-grade | | |
| particles (%) | | |

*Homogenization and milling using yttria-stabilized tetragonal zirconia beads having a diameter of 0.2 mm for 180 minutes.
**Nano-grade supernatant obtained after milling and centrifugation at a speed of 12,000 xg.

### Example 4

2.0 g of lecithin (HLB value: 8), 0.9 g of polysorbate 20 (HLB value: 16.7), and 0.7 g of sucrose stearate (HLB value: 15) (i.e., a total amount of 3.6 g (a concentration of 0.9% (w/v) relative to the volume of water)) were sequentially incorporated into 400 mL of water. After being homogenously stirred under heating, an aqueous solution comprising a complex stabilizer having an HLB value of 11.5 was obtained. 12 g (a concentration of 3% (w/v) relative to the volume of water) of curcumin was incorporated into the aqueous solution comprising a complex stabilizer. After stirring, a non-homogenously mixed liquid was obtained. The non-homogenously mixed liquid was subjected to a homogenization pretreatment using a homogenizer (Pro-400 Pro Scientific Inc.; speed: 6,000 rpm; size of the head of the homogenizer: 10×150 mm; homogenization time: 10 minutes), and then fed to a nano-grade wet grinder commercially available under the trade name “MiniCer” (manufactured and sold by Netzsch-Feinmahltechnik GmbH, Selb, Germany) in which the milling chamber was filled with 70% (v/v) of yttria-stabilized tetragonal zirconia beads with a diameter of 0.2 mm for circulation milling for 180 minutes. After milling, the aqueous dispersion was transferred to a centrifuge (Beckman J2-MC Centrifuge manufactured in U.S.A.) for centrifugation at a speed of 12,000×g at 25° C. for 10 minutes. The supernatant was collected to obtain an aqueous dispersion comprising curcumin. The particle size and concentration of curcumin in the aqueous dispersion was measured again. The result is shown in Table 4.

The dispersion was kept away from being exposed to light at 25° C. After storing for 4 months, particle size and concentration of the particles in the dispersion were measured. The result is shown in Table 4.

Table 4 shows that the freshly completed aqueous dispersion comprising curcumin had a particle size of 59±1 nm and a concentration of 12.13±1.71 mg/mL. After being stored for 4 Months, particle size slightly increased to 89±2 nm and the particles were still nano-grade, and concentration became 12.92±1.80 mg/mL. No significant change occurred. This shows that the dispersion comprising nano-curcumin had good storage stability.

TABLE 4

| | Particle size of | Concentration of |
|---|---|---|
| | the dispersion | the dispersion |
| | comprising | comprising |
| | nano-curcumin | nano-curcumin |
| Treatment | (nm) | (mg/mL) |
| Just prepared | 59 ± 1 | 12.13 ± 1.71 |
| Stored for 4 months | 89 ± 2 | 12.92 ± 1.80 |

### Example 5

2.0 g of lecithin (HLB value: 8) and 1.6 g of polysorbate 20 (HLB value: 16.7) (i.e., a total amount of 3.6 g (a concentration of 0.9% (w/v) relative to the volume of water)) were sequentially incorporated into 400 mL of water. After being homogenously stirred under heating, an aqueous solution comprising a complex stabilizer having HLB value of 12 was obtained. 24 g (a concentration of 6% (w/v) relative to the volume of water) of curcumin was incorporated into the aqueous solution comprising a complex stabilizer. After stirring, a non-homogenously mixed liquid was obtained. A portion of the non-homogenously mixed liquid was the non-homogenously mixed liquid obtained before and after centrifugation was subjected to testing for anti-inflammatory activity on cells as described in Example 6.

The non-homogenously mixed liquid was subjected to a homogenization pretreatment using a homogenizer (Pro-400 Pro Scientific Inc.; speed: 6,000 rpm; size of the head of the homogenizer: 10×150 mm; homogenization time: 10 minutes), and then fed to a nano-grade wet grinder commercially available under the trade name “MiniCer” (manufactured and sold by Netzsch-Feinmahltechnik GmbH, Selb, Germany) in which the milling chamber was filled with 70% (v/v) of yttria-stabilized tetragonal zirconia beads with a diameter of 0.2 mm for circulation milling for 180 minutes. A portion of the aqueous dispersion was subjected to testing for anti-inflammatory activity on cells. After milling, the aqueous dispersion was transferred to a centrifuge-(Beckman J2-MC Centrifuge manufactured in U.S.A.) for centrifugation at a speed of 12,000×g at 25° C. for 10 minutes. The supernatant was collected to obtain an aqueous dispersion comprising nano-curcumin. A portion of the aqueous dispersion was subjected to testing for anti-inflammatory activity on cells.

Before carrying out a test for determining the anti-inflammatory activity on Raw 264.7 cells, the non-homogenously mixed liquid obtained before homogenization and the dispersion obtained after milling were adjusted to the same concentration, 1 mg/mL. 1 μL of the liquid was added to a nutrient solution of cells to allow the dispersion of curcumin reaches an amount of 0.50% (v/v) on the basis of the total amount of the nutrient solution of cells. The influence of the dispersion on the generation of NO of Raw 264.7 cells was tested. The result is shown in Table 5.

Table 5 shows that the non-homogenously mixed liquid obtained before homogenization is difficult to inhibit the anti-inflammation which generates NO. After adding an aqueous dispersion comprising nano/submicron curcumin (before centrifugation) and an aqueous dispersion comprising nano/submicron curcumin (after centrifugation), NO inhibition rate of 77.01 and 100% were obtained respectively. The result shows that the dispersion obtained after milling was useful to provide an anti-inflammatory efficacy on Raw 264.7 cells. The solvent (Dimethyl sulfoxide) no longer requires.

The example shows that both of the aqueous dispersions of nano-grade and nano/submicron curcumin significantly enhanced the anti-inflammatory efficacy of cells.

TABLE 5

| | Rate of a mixed liquid or dispersion of | |
|---|---|---|
| | curcumin in inhibiting the generation of | |
| | NO from Raw 264.7 cells (%)** | |
| Treatment | Before centrifugation | After centrifugation* |
| Before | 0.00 ± 0.00 | 0.00 ± 0.00 |
| homogenization | | |
| After milling | 77.01 ± 2.35 | 99.89 ± 0.02 |

*The nano-grade supernatant obtained after milling and centrifugation at a speed of 12,000 xg.
**The concentration of each dispersion was adjusted to 1 mg/mL. 0.50% (v/v) of the dispersion was added to the culture solution of cells.

### Example 6

Hydrophobic, functional compounds, for example, silymarin, lutene, and isoflavonoid, also have anti-inflammatory activity. However, their anti-inflammatory activities are different. In Example 6, an anti-inflammatory test on cells was carried out using the macrophage, Raw 264.7 cells. The non-homogenously mixed liquid and nano/submicron dispersion used in this example were prepared according to the method of Example 3.

The non-homogenously mixed liquid and nano/submicron aqueous dispersion of silymarin, lutene, and isoflavonoid prepared according to the method of Example 3 were added to the culture solutions of Raw 264.7 cells at an appropriate concentration, respectively. The above functional substances added to the culture solutions of Raw 264.7 cells were 0.4, 2.0, and 0.4 μl, respectively, so that the concentrations of silymarin, lutene, and isoflavonoid were 0.2% (v/v), 1.0% (v/v), and 0.2% (v/v) on the basis of the total volume of the culture solutions of Raw 264.7 cells. The influence of the liquids comprising silymarin, lutene, and isoflavonoid on generation of NO from Raw 264.7 cells was tested. The result is shown in Table 6.

Table 6 shows that the non-homogenously mixed liquid and nano/submicron aqueous dispersion of silymarin, lutene, and isoflavonoid obtained before homogenization had no anti-inflammatory activity on Raw 264.7 cells. After milling, the resulting nano/submicron aqueous dispersion showed significant inhibition in NO generated from Raw 264.7 cells. The NO inhibition rates were 69.21, 40.42, and 35.56%, respectively. The results show that after being formulated into nano/submicron grade, the dispersion of silymarin, lutene, and isoflavonoid showed a significantly enhanced anti-inflammatory efficacy on cells.

The example shows that both of the aqueous dispersions of nano-grade and nano/submicron curcumin significantly enhanced the anti-inflammatory efficacy of cells.

TABLE 6

| | Rate of inhibiting the generation of NO from | | |
|---|---|---|---|
| | Raw 264.7 cells (%) | | |
| | silymarin | lutene | isoflavonoid |
| | The amount of nano/submicron aqueous | | |
| | dispersion added to the cell solution | | |
| Treatment | 0.2% (v/v) | 1% (v/v) | 0.2% (v/v) |
| Before | 0 ± 0 | 0 ± 0 | 0 ± 0 |
| homogenization | | | |
| After milling | 69.21 ± 3.26 | 40.42 ± 1.25 | 35.56 ± 2.34 |

### Example 7

Although many functional compounds have good biological efficacy, they usually have poor oral absorption and show poor bioavailability. These functional compounds include hydrophobic compounds (e.g., curcumin) and hydrophilic compounds (e.g., catechin) (see references 25 and 26).

2.58 g of lecithin (HLB value: 8) and 3.42 g of sucrose stearate (HLB value: 15) (i.e., a total amount of 6 g (a concentration of 1.5% (w/v) relative to the volume of water)) were sequentially incorporated into 400 mL of water. The materials were homogenously stirred via heating to form an aqueous solution of a complex stabilizer having HLB value of 12.40 g of curcumin (a concentration of 10% (w/v) relative to the volume of water) was incorporated into the aqueous solution of a complex stabilizer. After stirring, a non-homogenously mixed liquid was obtained. The non-homogenously mixed liquid was subjected to a homogenization pretreatment using a homogenizer (Pro-400 Pro Scientific Inc.; speed: 6,000 rpm; size of the head of the homogenizer: 10×150 mm; homogenization time: 10 minutes), and then fed to a nano-grade wet grinder commercially available under the trade name “MiniCer” (manufactured and sold by Netzsch-Feinmahltechnik GmbH, Selb, Germany) in which the milling chamber was filled with 70% (v/v) of yttria-stabilized tetragonal zirconia beads with a diameter of 0.2 mm for circulation milling for 180 minutes. The aqueous dispersion of nano/submicron grade curcumin obtained after milling was provided for an animal test. The mixed liquid of curcumin used as a control group in this example was prepared by mixing curcumin and water without any stabilizer, and subjecting the materials to a homogenizer for homogenization.

The mixed liquid of curcumin and the aqueous dispersion of nano/submicron grade curcumin were fed to ICR mice in a feed amount of 0.2 g/kg of body weight and 2.5 g/kg of body weight. The mice were sacrificed 15, 30, 45, 60, 120, and 300 minutes after feeding. Their plasma was collected and treated with sulfatase for 2 hours to allow the relevant metabolites of curcumin in plasma to transform into curcumin. High performance liquid chromatography was used to analyze the amount of curcumin in plasma. The result is shown in Table 7.

Table 7 shows that for the mice that had taken a feed amount of 0.2 g/kg of body weight, the highest value of plasma concentration (Cₘₐₓ) and the area under plasma concentration-time curve (AUC) of the mice fed with a nano/submicron aqueous dispersion were 12.62 and 35.16 times those of the mice which had been fed with a mixed liquid of curcumin. For the mice that had taken a feed amount of 2.5 g/kg of body weight, the Cₘₐₓ and the AUC of the mice fed with a nano/submicron aqueous dispersion were 6.96 and 6.82 times those of the mice fed with a mixed liquid of curcumin. AUC stands for oral bioavailability. The results show that the bioavailability of rodents after oral administration of a nano/submicron aqueous dispersion increases 7-35 fold.

This example shows that the aqueous dispersion comprising nano/submicron grade curcumin prepared by the present invention has improved absorption. Accordingly, in addition to improving the dispersibility of curcumin in water, the present invention also improves absorption after oral administration.

TABLE 7

| | Feeding dose | Cₘₐₓ* | Tₘₐₓ** | AUC*** |
|---|---|---|---|---|
| Type of liquid fed | (g/kg bw) | (μg/mL) | (min) | (min × μg/mL) |
| Mixed liquid of curcumin | 0.2 | 0.47 ± 0.33 | 45 ± 11 | 36 ± 12 |
| Aqueous dispersion of | 0.2 | 5.96 ± 0.72 | 75 ± 42 | 1,250 ± 56 |
| nano/submicron grade curcumin | | | | |
| Times (nano/submicron grade | | 12.62 | — | 35.16 |
| dispersion/Mixed liquid) | | | | |
| Mixed liquid of curcumin | 2.5 | 1.83 ± 0.19 | 48 ± 7 | 276 ± 21 |
| Aqueous dispersion of | 2.5 | 12.70 ± 1.01 | 55 ± 7 | 1,884 ± 57 |
| nano/submicron grade curcumin | | | | |
| Folds (nano/submicron grade | | 6.96 | — | 6.82 |
| dispersion/Mixed liquid) | | | | |

*Cₘₐₓ: The highest value of concentration in plasma.
**Tₘₐₓ: The time when the highest value of concentration in plasma is reached.
***AUC: The area under plasma concentration-time curve calculated by using WinNonLin and non-compartmental model.

### Example 8

From the above example, it is known that an aqueous dispersion comprising nano/submicron grade curcumin has better absorption and anti-inflammatory activity. This example proves that an aqueous dispersion comprising nano/submicron grade curcumin has better oral anti-inflammatory activity.

The mixed liquid of curcumin and the aqueous dispersion comprising nano/submicron grade curcumin of this example were prepared according to the method of Example 7.

0.8 mL of the mixed liquid of curcumin and the aqueous dispersion comprising nano/submicron grade curcumin were fed to ICR mice respectively. After half an hour, TPA (Phorbol 12-myristate 13-acetate) was used to induce inflammatory symptoms on the ears of mice. The mice were sacrificed after 6 hours. Round pieces (a diameter of 6 mm) of the ears were cut and weighed. The results are shown in Table 8.

Table 8 shows that feeding an aqueous dispersion comprising nano/submicron grade curcumin inhibited 36.17% of edema and showed a significant difference (P<0.05). For the mice fed with the mixed liquid of curcumin, no efficacy against edema was found. The result shows that oral administration of an aqueous dispersion comprising nano/submicron grade curcumin provides significant anti-inflammatory activity.

TABLE 8

| | Average weight | Inhibition |
|---|---|---|
| | of each ear | ratio |
| Treatment | mg ± SE | (%) |
| Feeding water and topical with acetone | 10.15 ± 0.46 | — |
| Feeding water and topical with TPA | 20.49 ± 0.47 | — |
| Feeding a mixed liquid of curcumin and | 20.14 ± 1.35 | 3.38 |
| topical with TPA | | |
| Feeding nano/submicron grade curcumin | 16.75 ± 0.99* | 36.17* |
| solution and topical with TPA | | |

*Calculated by Student's test, showing a significant different of P < 0.05.

### REFERENCES

1. X. Wang, Y. Jiang, Y. W. Wang, M. T. Huang, C. T. Ho and Q. Huang: Enhancing anti-inflammation activity of curcumin through O/W nano-emulsions. Food Chem. 108: 419-424 (2008).
2. C. C. Lin, H. Y. Lin, H. C. Chen, M. W. Yu and M. H. Lee: Stability and characterization of phospholipids-based curcumin-encapsulated micro emulsions. Food Chem. 116: 923-928 (2009).
3. L. Jia, H. Wong, C. Cerna, and S. D. Weisman: Effect of nanonization on absorption of 301029: ex vivo and in vim pharmacokinetic correlations determined by liquid chromatography/mass spectrometry. Pharmaceutical Res. 19(8): 1091-1096 (2002).
4. A. Giori: Phospholipid complexes of curcumin having improved bioavailability. World Intellectual Property Organization patent WO 101551 A2 (2007).
5. A. Giori: Phospholipid complexes of curcumin having improved bioavailability. EP patent 1 837 030 A1 (2007).
6. A. Giori: Phospholipid complexes of curcumin having improved bioavailability. U.S. Pat. No. 0,131,373 A1. (2009)
7. T. H. Marczylo, R. D. Verschoyle, D. N. Cooke, P. Morazzoni, W. P. Steward and P. Mprazzoni: Comparison of systemic availability of curcumin with that of curcumin formulated with phosphatidylcholine. Cancer Chemother Pharmacol. 60: 171-177 (2007).
8. Nagahama Touru, Hasegawa Kazuo, Nakajima Toshiaki, Itou Yuuji: Microdrop emulsions. China patent application no. 97194863.1 (CN1098120C).
9. K. Maiti, K. Mukherjee, A. Gantait, B. P. Saha, P. K. Mukherjee: Curcumin-phospholipid complex: Preparation, therapeutic evaluation and pharmacokinetic study in rats. Inter. J. Pharm. 330: 155-163 (2007).
10. J. Shaikh, D. D. Ankola, V. Beniwal, D. Singh, M. N. V. Ravi Kumar: Nanoparticle encapsulation improves oral bioavailability of curcumin by at least 9-fold when compared to curcumin administered with piperine as absorption enhancer. European J. Pharmaceutical Sci. 37: 223-230 (2009).
11. W. Tiyaboonchai, W. Tungpradit, and P. Plianbangchang: Formulation and characterization of curcuminoids loaded solid lipid nanoparticles. Inter. J. Pharm. 337: 299-306 (2007).
12. S. Bisht, G. Feldmann, S. Soni, R. Ravi, C. Karikar, A. Maitra and A. Maitra: Polymeric nanoparticle-encapsulated curcumin (“nanocurcumin”): a novel strategy for human cancer therapy. J. Nanobiotechnology 5: 3-20 (2007).
13. Liang Wei et al, “Pharmaceutical composition for improving the solubility and bioavailability of curcimun and method for preparing the same;” China patent application no. 200310118422.0 (Publication No. CN1274300C).
14. Lou Hong-Shing et. al, “Complex of curcimun and phospholipid and method for preparing the same;” China patent application no. 200410036402.3 (Publication No. CN1283237C).
15. Liu Jei et. al, “Control-release fine particles of nano-curcumin and method for preparing the same;” China patent application no. 200610125179.9 (Publication No. CN1957926A).
16. Fu Shu Wen et. al. “Methods for preparing a drug-lipid complex and containing drugs;” Taiwan patent publication no. 200533387.
17. L. Jia, H. Wong, Y. Wang, M. Garza. S. D. Weitman: Carbendazim: disposition, cellular permeability, metabolite identification, and pharmacokinetic comparison with its nanoparticle. J. Pharmaceutical Sci. 92(1): 161-171 (2003)
18. Lin Kuan Ting: Anti-oxidation foods and health, published by chemical industries, Bei-Jen, China (2004).
19. J. Cui, B. Yu, Y. Zhao, W. Zhu, H. Li, H. Lou and G. Zhai: Enhancement of oral absorption of curcumin by self-microemulsifying drug delivery systems. Internation J. Pharmaceutics 371: 148-155 (2009).
20. P. Anand, A. B. Kunnumakkara, R. A. Newman and R. R. Aggarwal: Bioavailability of curcumin: problems and promises. Mol. Pharm. 4: 807-818 (2007).
21. H. P. T. Ammon and M. A. Wahl: Pharmacology of Curcuma longa. Planta Med. 57: 1-7 (1991).
22. D. Suresh and K. Srinivassan: Studies on the in vitro absorption of spice principles—Curcumin, capsaicin and piperine in rat intestines. Food and Chemical Toxicology 45:1437-1442 (2007).
23. M. Ĉiz. M. G. Pavelkova, L. Kralova, J. L. Kubala, and A. Lojik: The influence of wine polyphenols on reactive oxygen and nitrogen species production by murine macrophages RAW 264.7. Physiol. Res. 57: 393-402 (2008).
24. H. K. Kim, B. S. Cheon, Y. H. Kim, S. Y. Kim and H. P. Kim: Effects of naturally occurring flavonoids on nitric oxide production in the macrophage cell line RAW 264.7 and their structure-activity relationships. Biochem Pharmacol 58:759-65 (1999).
25. A. Dube, J. A. Nicolazzo and I. Larson: Chitosan nanoparticles enhance the intestinal absorption of the green tea catechins(+)-catechin and (−)-epigallocatechin gallate. Eur J. Pharm Sci. 41:219-225 (2010).
26. S. Shugarts and L. Z. Benet: The role of transporters in the pharmacokinetics of orally administered drugs. Pharm Res 26:2039-2054 (2009).

What is claimed is:

1. A process for preparing an aqueous dispersion containing a high concentration of nano/submicron, hydrophobic, functional compounds, which comprises the following steps:
formulating a complex stabilizer and water into an aqueous solution containing a complex stabilizer, wherein said complex stabilizer has an HLB value of about 10 to about 17 and comprises lecithin and at least one non-phospholipid selected from polysorbate, sucrose ester, and polyglycerol fatty acid ester,
incorporating hydrophobic, functional compounds into the aqueous solution containing a complex stabilizer to form a non-homogenously mixed liquid wherein the weight ratio of the hydrophobic, functional compounds to the complex stabilizer is from 2:1 to 10:1,
subjecting the non-homogenously mixed liquid to a homogenization pretreatment to form a homogenously mixed liquid,
subjecting the homogenously mixed liquid to nano-grade wet grinding to form an aqueous dispersion, and
optionally, subjecting the aqueous dispersion from nano-grade wet grinding to a centrifugal step and collecting the supernatant.

2. The process of claim 1, wherein the hydrophobic, functional compounds are selected from the group consisting of lipid-soluble vitamins, carotenenoid terpenoids, non-flavonoides polyphenolics, flavonoides polyphenolics, and mixtures thereof.

3. The process of claim 2, wherein the hydrophobic, functional compounds are selected from the group consisting of vitamin A, vitamin D, vitamin E, vitamin K, CoQ10, lycopene, carotene, lutene, zeaxanthin, curcumin, silymarin, isoflavonoid, hesperidin, seasamin, and mixtures thereof.

4. The process of claim 1, wherein the complex stabilizer has an HLB value of about 10 to about 15.

5. The process of claim 1, wherein the aqueous solution containing a complex stabilizer is prepared by a method comprising separately melting lecithin and at least one non-phospholipid in a weight ratio of about 1:99 to about 99:1 via heating, homogenously stirring the non-phospholipid and the lecithin to form a complex stabilizer having an HLB value of about 10 to about 17, incorporating the complex stabilizer into water in an amount of about 0.01% to about 10.0% (w/v) relative to the volume of water; and homogenously stirring it.

6. The process of claim 5, wherein the complex stabilizer is incorporated into water in an amount of about 0.1% to about 4.5% (w/v) relative to the volume of water.

7. The process of claim 1, wherein the aqueous solution containing a complex stabilizer is prepared by a method comprising separately incorporating lecithin and at least one non-phospholipid in a weight ratio of about 1:99 to about 99:1 into water in a total amount of the lecithin and the non-phospholipid of about 0.01% to about 10.0% (w/v) relative to the volume of water, and homogeneously stirring it.

8. The process of claim 7, wherein the complex stabilizer is incorporated into water in an amount of about 0.1% to about 4.5% (w/v) relative to the volume of water.

9. The process of claim 1, wherein the weight ratio of the hydrophobic, functional compounds to the complex stabilizer is about 3:1 to about 8:1.

10. The process of claim 1, wherein the homogenization pretreatment is carried out by using a homogenizer or an ultrasonic processor.

11. The process of claim 1, wherein the nano-grade wet grinding miller uses yttria-stabilized tetragonal zirconia beads having a diameter of 0.05-1.0 mm, the milling time is about 5 to about 300 minutes, and the speed is about 600 to about 4,000 rpm.

12. An aqueous dispersion containing a high concentration of nano/submicron, hydrophobic, functional compounds prepared from the process of claim 1.

13. The aqueous dispersion of claim 12, wherein the concentration of nano/submicron, hydrophobic, functional compounds in the aqueous dispersion containing a high concentration of nano/submicron, hydrophobic, functional compounds is from about 1 mg/mL (0.1%) to about 200 mg/mL (20%).

14. The aqueous dispersion of claim 13, wherein the concentration of nano/submicron, hydrophobic, functional compounds in the aqueous dispersion containing a high concentration of nano/submicron, hydrophobic, functional compounds is from about 10 mg/mL (1%) to about 150 mg/mL (15%).

15. The aqueous dispersion of claim 12, wherein the nanoparticles are present in a proportion of about 20% to about 85% (w/w) on the basis of the total particles.

16. The aqueous dispersion of claim 15, wherein the nanoparticles are present in a proportion of about 40% to about 85% (w/w) on the basis of the total particles.

17. The aqueous dispersion of claim 16, wherein the nanoparticles are present in a proportion of about 60% to about 85% (w/w) on the basis of the total particles.

18. The aqueous dispersion of claim 13, wherein the nanoparticles are present in a proportion of about 20% to about 85% (w/w) on the basis of the total particles.

19. The aqueous dispersion of claim 18, wherein the nanoparticles are present in a proportion of about 40% to about 85% (w/w) on the basis of the total particles.

20. The aqueous dispersion of claim 19, wherein the nanoparticles are present in a proportion of about 60% to about 85% (w/w) on the basis of the total particles.

21. The aqueous dispersion of claim 14, wherein the nanoparticles are present in a proportion of about 20% to about 85% (w/w) on the basis of the total particles.

22. The aqueous dispersion of claim 21, wherein the nanoparticles are present in a proportion of about 40% to about 85% (w/w) on the basis of the total particles.

23. The aqueous dispersion of claim 22, wherein the nanoparticles are present in a proportion of about 60% to about 85% (w/w) on the basis of the total particles.
